(19) United States
(12) Patent Application Publication
(10) Pub. No.: US 2024/0363244 A1
(43) Pub. Date: Oct. 31, 2024

(54) METHOD AND DEVICE FOR PREDICTING STENOSIS OF DIALYSIS ACCESS BY USING CONVOLUTIONAL NEURAL NETWORK

(71) Applicants: UIF (University Industry Foundation), Yonsei University, Seoul (KR); SEOUL NATIONAL UNIVERSITY HOSPITAL, Seoul (KR)

(72) Inventors: Jong Yun WON, Seoul (KR); Ki Chang HAN, Seoul (KR); Jae Hyon PARK, Seoul (KR); In Sun PARK, Seongnam-si, Gyeonggi-do (KR)

(21) Appl. No.: 18/560,370

(22) PCT Filed: May 13, 2022
(86) PCT No.: PCT/KR2022/006887

§ 371 Date: Nov. 10, 2023

(30) Foreign Application Priority Data
May 13, 2021 (KR) 10-2021-0062071

(51) Int. Cl. G16H 50/20; G16H 10/60; G16H 50/50

(52) U.S. Cl. CPC G16H 50/20; G16H 10/60; G16H 50/50

(57) ABSTRACT

A method and a device for predicting stenosis of a dialysis access by using a convolutional neural network (CNN) according to a preferable embodiment of the present invention can predict, from audio data with respect to a dialysis access of an object, a degree of stenosis of the dialysis access on the basis of a stenosis prediction model including a convolutional neural network so as to predict the degree of stenosis of the dialysis access more precisely and guide an additional examination and treatment according thereto.

### TECHNICAL FIELD

The present invention relates to a method and a device for predicting stenosis of a dialysis access by using a convolutional neural network, and more particularly, to a method and device for diagnosing a dialysis access stenosis requiring treatment.

### BACKGROUND ART

Abnormalities of dialysis access, such as arteriovenous fistulas are identified by relying heavily on palpation and auscultation. Actually, thrill and pulsation felt during palpation before and after stenosis may greatly vary depending on the area. Thrill during the palpation may be heard as a vibration sound such as high pitch bruit in an audible frequency range when using a stethoscope and similarly, the presence and the intensity of bruit may indirectly diagnose the stenosis and occlusion of the arteriovenous fistula. However, there are many subjective factors involved in assessing auscultation, making it difficult to objectively identify meaningful stenosis which require treatment such as angioplasty.

### DISCLOSURE

### Technical Problem

An object to be achieved by the present invention is to provide a method and a device for predicting a stenosis of a dialysis access by using a convolutional neural network which predict a degree of stenosis of the dialysis access, from audio data with respect to a dialysis access of an object, on the basis of a stenosis prediction model including a convolutional neural network.

Other and further objects of the present disclosure which are not specifically described can be further considered within the scope easily deduced from the following detailed description and the effect.

### Technical Solution

In order to achieve the above-described object, a method for predicting stenosis of a dialysis access by using a convolutional neural network according to a preferable embodiment of the present invention includes acquiring audio data with respect to a dialysis access of an object; and predicting a degree of stenosis corresponding to the audio data on the basis of a stenosis prediction model including a previously learned convolutional neural network (CNN).

Here, the acquiring of audio data is configured by preprocessing the audio data and the predicting of a degree of stenosis is configured by inputting the preprocessed audio data to the stenosis prediction model and predicting a degree of stenosis corresponding to the audio data on the basis of an output value of the stenosis prediction model.

Here, the acquiring of audio data is configured by acquiring the audio data in a predetermined interval from the audio data, acquiring a spectrogram on the basis of the audio data in the predetermined interval, normalizing the acquired spectrogram, and adjusting a size of the normalized spectrogram.

Here, the method may further include learning the stenosis prediction model on the basis of a learning data set including first audio data with respect to the dialysis access acquired before a procedure and second audio data with respect to the dialysis access acquired after the procedure.

Here, the stenosis prediction model has the spectrogram as an input and a degree of the stenosis as an output.

Here, the learning of the stenosis prediction model is configured by preprocessing the learning data set and learning the stenosis prediction model on the basis of the learning data set preprocessed with first audio data as a first correct answer label and second audio data as a second correct answer label.

Here, the learning of the stenosis prediction model is configured by acquiring the audio data in a predetermined interval from the audio data, with respect to audio data included in the learning data set, acquiring a spectrogram on the basis of the audio data in the predetermined interval, normalizing the acquired spectrogram, horizontally shifting the normalized spectrogram to increase the number, and adjusting the size of the increased spectrogram to preprocess the learning data set.

Here, the learning of the stenosis prediction model is configured by dividing the preprocessed learning data set into a training data set, a tuning data set, and a validation data set according to a predetermined criteria, learning the stenosis prediction model by using the training data set, tuning the learned stenosis prediction model using the tuning data set, and validating the tuned stenosis prediction model using the validation data set.

In order to achieve the above-described technical object, a computer program according to a preferable embodiment of the present invention is stored in a computer readable storage medium to allow a computer to execute any one of the methods for predicting a stenosis of a dialysis access by using a convolutional neural network.

In order to achieve the above-described object, a device for predicting stenosis of a dialysis access by using a convolutional neural network according to a preferable embodiment of the present invention is a device for predicting a stenosis of a dialysis access by using a convolutional neural network (CNN) including a memory which stores one or more programs to predict a stenosis of a dialysis access using a convolutional neural network (CNN); and one or more processors which perform an operation for predicting a stenosis of a dialysis access using a convolutional neural network (CNN) according to one or more program stored in the memory, the processor acquires audio data with respect to a dialysis access of an object and predicts a degree of stenosis corresponding to the audio data on the basis of the stenosis prediction model including a previously learned convolutional neural network (CNN).

Here, the processor preprocesses the audio data, inputs the preprocessed audio data to the stenosis prediction model, and predicts a degree of stenosis corresponding to the audio data on the basis of an output value of the stenosis prediction model.

Here, the processor learns the stenosis prediction model on the basis of a learning data set including first audio data with respect to the dialysis access acquired before a procedure and second audio data with respect to the dialysis access acquired after the procedure.

Here, the processor preprocesses the learning data set and learns the stenosis prediction model on the basis of the learning data set preprocessed with first audio data as a first correct answer label and second audio data as a second correct answer label.

### Advantageous Effects

According to the method and the device for predicting a stenosis of a dialysis access by using a convolutional neural network according to a preferable embodiment of the present invention, a degree of stenosis of the dialysis access is predicted from audio data with respect to a dialysis access of an object on the basis of a stenosis prediction model including a convolutional neural network to more precisely predict the degree of stenosis of the dialysis access and guide an additional examination and treatment according thereto.

The effects of the present disclosure are not limited to the technical effects mentioned above, and other effects which are not mentioned can be clearly understood by those skilled in the art from the following description.

### DESCRIPTION OF DRAWINGS

FIG. 1 is a block diagram of a device for predicting a stenosis of a dialysis access by using a convolutional neural network according to a preferable embodiment of the present invention.

FIG. 2 is a flowchart of a method for predicting a stenosis of a dialysis access by using a convolutional neural network according to a preferable embodiment of the present invention.

FIG. 3 is a view for explaining a learning process of a stenosis prediction model according to a preferable embodiment of the present invention.

FIG. 4 is a view for explaining a pre-processing process of a learning data set illustrated in FIG. 3.

FIG. 5 is a view for explaining a process of predicting a degree of stenosis using a stenosis prediction model according to a preferable embodiment of the present invention.

FIG. 6 is a view for explaining a pre-processing process of audio data illustrated in FIG. 5.

FIG. 7 is a view for explaining an example of a process of learning a stenosis prediction model and a process of predicting a degree of stenosis according to a preferable embodiment of the present invention.

FIG. 8 is a view for explaining an example of a process of acquiring a spectrogram according to a preferable embodiment of the present invention.

FIG. 9 is a view illustrating an example of a spectrogram acquired by the process illustrated in FIG. 8.

FIG. 10 is a view illustrating an example of a spectrogram acquired by the process illustrated in FIG. 8 in which FIG. 10(a) illustrates a spectrogram acquired on the basis of audio data with respect to a dialysis access acquired before a procedure and FIG. 10(a) illustrates a spectrogram acquired on the basis of audio data with respect to a dialysis access acquired after a procedure.

FIG. 11 is a view for explaining a performance of a stenosis prediction model according to a preferable embodiment of the present invention in which FIG. 11(a) illustrates a confusion matrix and FIG. 11(b) illustrates a receiver operation characteristic (ROC) curve.

### BEST MODE

Hereinafter, embodiments of the present invention will be described in detail with reference to the accompanying drawings. Advantages and characteristics of the present invention and a method of achieving the advantages and characteristics will be clear by referring to preferable embodiments described below in detail together with the accompanying drawings. However, the present invention is not limited to preferable embodiments disclosed herein, but will be implemented in various different forms. The preferable embodiments are provided by way of example only so that a person of ordinary skilled in the art can fully understand the disclosures of the present invention and the scope of the present invention. Therefore, the present invention will be defined only by the scope of the appended claims. Like reference numerals generally denote like elements throughout the specification.

Unless otherwise defined, all terms (including technical and scientific terms) used in the present specification may be used as the meaning which may be commonly understood by the person with ordinary skill in the art, to which the present disclosure belongs. It will be further understood that terms defined in commonly used dictionaries should not be interpreted in an idealized or excessive sense unless expressly and specifically defined.

In the specification, the terms “first” and “second” are used to distinguish one component from the other component so that the scope should not be limited by these terms. For example, a first component may also be referred to as a second component and likewise, the second component may also be referred to as the first component.

In the present specification, in each step, numerical symbols (for example, a, b, and c) are used for the convenience of description, but do not explain the order of the steps so that unless the context apparently indicates a specific order, the order may be different from the order described in the specification. That is, the steps may be performed in the order as described or simultaneously, or an opposite order.

In this specification, the terms “have”, “may have”, “include”, or “may include” represent the presence of the characteristic (for example, a numerical value, a function, an operation, or a component such as a part”), but do not exclude the presence of additional characteristic.

Hereinafter, a preferable embodiment of a method and a device for predicting a stenosis of a dialysis access by using a convolutional neural network according to the present invention will be described in detail with reference to the accompanying drawings.

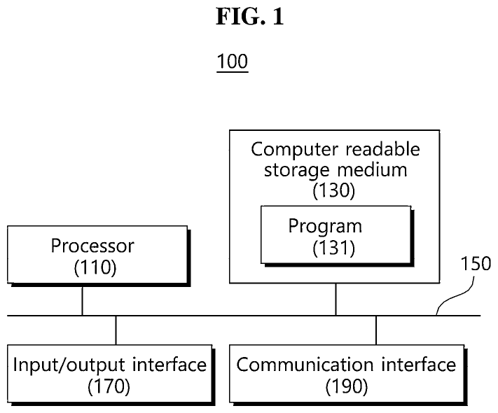

First, a device for predicting a stenosis of a dialysis access by using a convolutional neural network according to a preferable embodiment of the present invention will be described with reference to FIG. 1.

FIG. 1 is a block diagram of a device for predicting a stenosis of a dialysis access by using a convolutional neural network according to a preferable embodiment of the present invention.

Referring to FIG. 1, a device for predicting a stenosis of a dialysis access by using a convolutional neural network according to a preferable embodiment of the present invention may predict a degree of stenosis of the dialysis access from audio data with respect to a dialysis access (arteriovenous fistula) of an object on the basis of a stenosis prediction model including a convolutional neural network (CNN).

To this end, the stenosis predicting device 100 may include one or more processors 110, a computer readable storage medium 130, and a communication bus 150.

The processor 110 controls the stenosis predicting device 100 to operate. For example, the processor 110 may execute one or more programs 131 stored in the computer readable storage medium 130. One or more programs 131 include one or more computer executable instructions and when the computer executable instruction is executed by the processor 110, the computer executable instruction may be configured to allow the stenosis predicting device 100 to perform an operation for predicting a stenosis of a dialysis access by using a convolutional neural network (CNN).

The computer readable storage medium 130 is configured to store a computer executable instruction or program code, program data and/or other appropriate format of information to predict a stenosis of a dialysis access by using a convolutional neural network (CNN). The program 131 stored in the computer readable storage medium 130 includes a set of instructions executable by the processor 110. In one preferable embodiment, the computer readable storage medium 130 may be a memory (a volatile memory such as a random access memory, a non-volatile memory, or an appropriate combination thereof), one or more magnetic disk storage devices, optical disk storage devices, flash memory devices, and another format of storage media which are accessed by the stenosis predicting device 100 and store desired information, or an appropriate combination thereof.

The communication bus 150 interconnects various other components of the stenosis predicting device 100 including the processor 110 and the computer readable storage medium 130 to each other.

The stenosis predicting device 100 may include one or more input/output interfaces 170 and one or more communication interfaces 190 which provide an interface for one or more input/output devices. The input/output interface 170 and the communication interface 190 are connected to the communication bus 150. The input/output device (not illustrated) may be connected to another components of the stenosis predicting device 100 by means of the input/output interface 170.

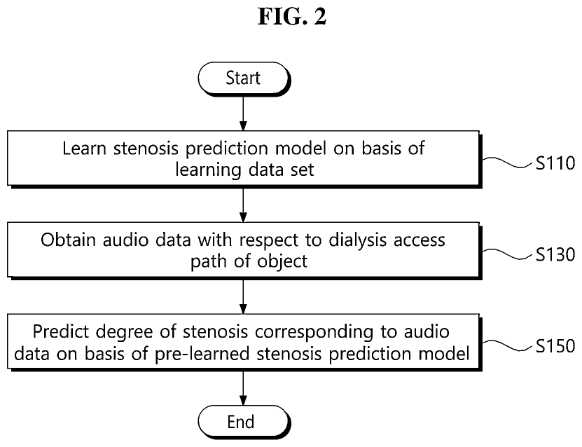

Now, a method for predicting a stenosis of a dialysis access by using a convolutional neural network according to a preferable embodiment of the present invention will be described with reference to FIGS. 2 to 6.

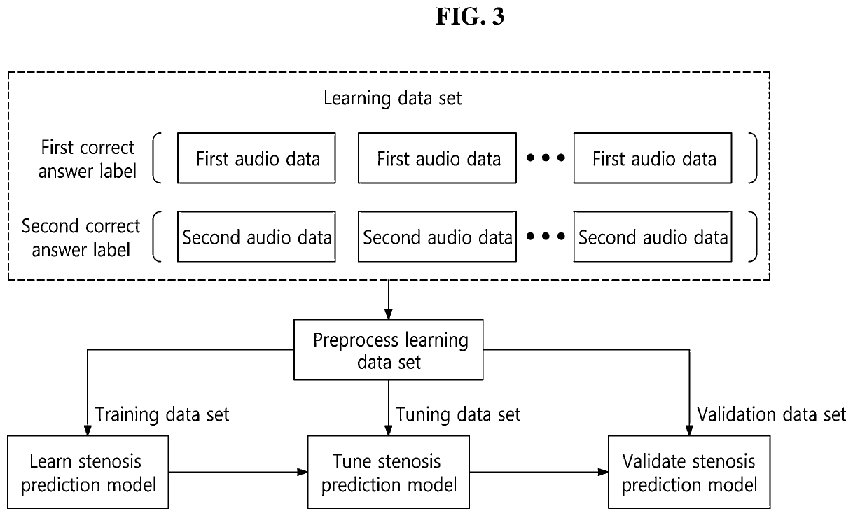

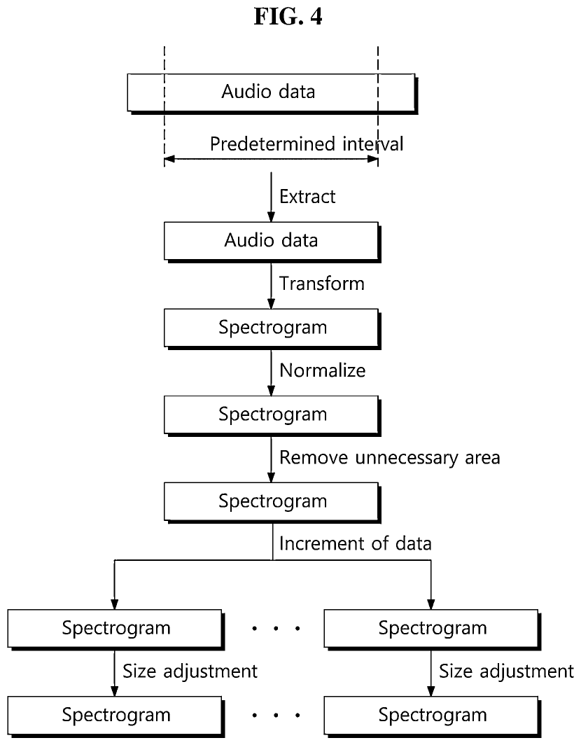

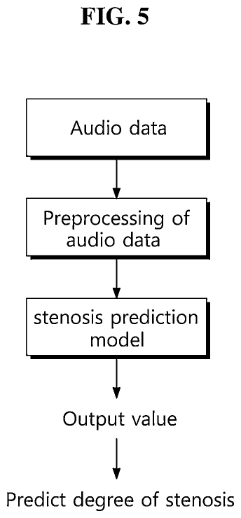

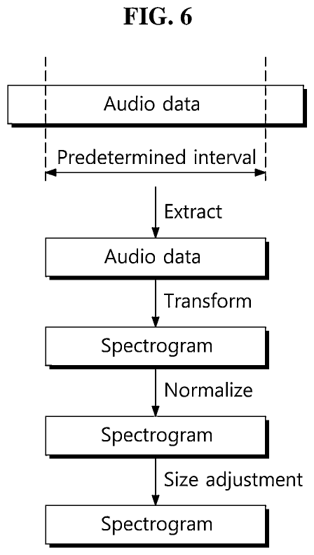

FIG. 2 is a flowchart of a method for predicting a stenosis of a dialysis access by using a convolutional neural network according to a preferable embodiment of the present invention, FIG. 3 is a view for explaining a learning process of a stenosis prediction model according to a preferable embodiment of the present invention, FIG. 4 is a view for explaining a pre-processing process of a learning dataset illustrated in FIG. 3, FIG. 5 is a view for explaining a process of predicting a degree of stenosis using a stenosis prediction model according to a preferable embodiment of the present invention, and FIG. 6 is a view for explaining a pre-processing process of audio data illustrated in FIG. 5.

Referring to FIG. 2, the processor 110 of the stenosis predicting device 100 may learn a stenosis prediction model on the basis of learning data set.

Here, the stenosis prediction model includes a convolutional neural network (CNN) and has a spectrogram as an input and a degree of stenosis as an output. For example, the degree of stenosis is a value indicating that a degree of stenosis of the dialysis access is 50% or higher and may have a value between 0 and 1.

The learning data set includes first audio data with respect to a dialysis access acquired before the angioplasty procedure and second audio data with respect to a dialysis access acquired after the angioplasty procedure. For example, audio data (a sound of an audible frequency band between 20 Hz to 1000 Hz) for the patient's dialysis access (arteriovenous fistulas) is acquired using an electronic stethoscope prior to performing the angioplasty. Similarly, after performing the angioplasty, audio data of the patient's dialysis access (arteriovenous fistulas) is acquired using an electronic stethoscope.

For example, as illustrated in FIG. 3, the processor 110 learns a final stenosis prediction model by going through “a process of preprocessing a learning data set”->“a process of learning a stenosis prediction model”->“a process of tuning a stenosis prediction model”->“a process of validating a stenosis prediction model”.

That is, the processor 110 preprocesses the learning data set.

To be more specific, referring to FIG. 4, the processor 110 may preprocess audio data included in the learning data set by going through the following process.

The processor 110 acquires audio data in a predetermined interval of audio data. For example, the processor 110 may extract audio data in a predetermined interval (2 seconds to 8 seconds) to remove effects of a noise.

The processor 110 acquires a spectrogram on the basis of audio data in a predetermined interval. For example, the processor 110 may convert audio data into a spectrogram using Fourier transform (FT).

The processor 110 normalizes the acquired spectrogram.

The processor 110 removes unnecessary areas (an edge or a boundary area) from the normalized spectrogram prior to performing data augmentation.

The processor 110 horizontally shifts the normalized spectrogram to increase the number of spectrograms. For example, the spectrogram 110 horizontally shifts the spectrograms multiple times on a time axis to increase the number of spectrograms.

The processor 110 may adjust an increased size of the spectrograms. For example, the processor 110 adjusts the size of the spectrogram to reduce the size of the spectrogram to a predetermined size (for example, 512×512).

Next, the processor 110 learns the stenosis prediction model on the basis of a preprocessed learning data set with first audio data as a first correct answer label and second audio data as a second correct answer label.

Here, the first correct answer label represents a state in which a degree of stenosis of the dialysis access is 50% or higher and for example, may be set to “1”. Here, the second correct answer label represents a state in which a degree of stenosis of the dialysis access is less than 50% and for example, may be set to “0”.

To be more specific, the processor 110 learns the stenosis prediction model by going through the following process, on the basis of the preprocessed learning data set.

The processor 110 may divide the preprocessed learning data set into a training data set, a tuning data set, and a validation data set on the basis of the predetermined criteria. For example, the processor 110 divides the first audio data set of the learning data set into a training data set, a tuning data set, and a validation data set according to a predetermined ratio “7:2:1” and divides the second audio set of the learning data set into a training data set, a tuning data set, and a validation data set.

The processor 110 learns the stenosis prediction model using a training data set.

The processor 110 tunes the learned stenosis prediction model using the tuning data set.

The processor 110 validates the tuned stenosis prediction model using the validation data set.

Thereafter, the processor 110 acquires audio data with respect to the dialysis access of the object (S130).

For example, as illustrated in FIG. 5, the processor 110 acquires the audio data by going through “a process of acquiring audio data”->“a process of preprocessing audio data”.

That is, the processor 110 acquires audio data with respect to the dialysis access of the object. For example, audio data (a sound in an audible frequency band between 20 Hz to 1000 Hz) for the patient's dialysis access whose degree of stenosis is determined is acquired using an electronic stethoscope.

The processor 110 may preprocess the acquired audio data.

To be more specific, referring to FIG. 6, the processor 110 may preprocess audio data by going through the following process.

The processor 110 acquires audio data in a predetermined interval of audio data. For example, the processor 110 may extract audio data in a predetermined interval (2 seconds to 8 seconds) to remove effects of a noise.

The processor 110 acquires a spectrogram on the basis of audio data in a predetermined interval. For example, the processor 110 may convert audio data into a spectrogram using Fourier transform (FT).

The processor 110 normalizes the acquired spectrogram.

The processor 110 may adjust a size of the normalized spectrograms. For example, the processor 110 adjusts the size of the spectrogram to reduce the size of the spectrogram to a predetermined size (for example, 512×512).

Thereafter, the processor 110 predicts the degree of stenosis corresponding to the audio data on the basis of the previously learned stenosis prediction model (S150).

For example, as illustrated in FIG. 5. The processor 110 predicts the degree of stenosis of the dialysis access of the object by going through “a process of inputting preprocessed audio data”->“a process of acquiring an output value of a stenosis prediction model”->“a process of predicting a degree of stenosis”.

That is, the processor 110 inputs the preprocessed audio data to the stenosis prediction model.

The processor 110 may predict the degree of stenosis corresponding to audio data on the basis of an output value of the stenosis prediction model.

For example, when the output value (that is, a degree of stenosis) of the stenosis prediction model is “0.95”, it means that a probability of a degree of stenosis of the dialysis access of the object of 50% or higher is 95%. Accordingly, the processor 110 may predict the degree of stenosis of the object to be 95%.

The processor 110 compares the output value (that is, a degree of stenosis) of the stenosis prediction model with a predetermined threshold (for example, 0.5). When the output value (that is, the degree of stenosis) is equal to or higher than the threshold, the processor 110 predicts the degree of stenosis of the object to be “suspected stenosis” and when the output value (that is, the degree of stenosis) is lower than the threshold, predicts the degree of stenosis of the object to be “no stenosis”.

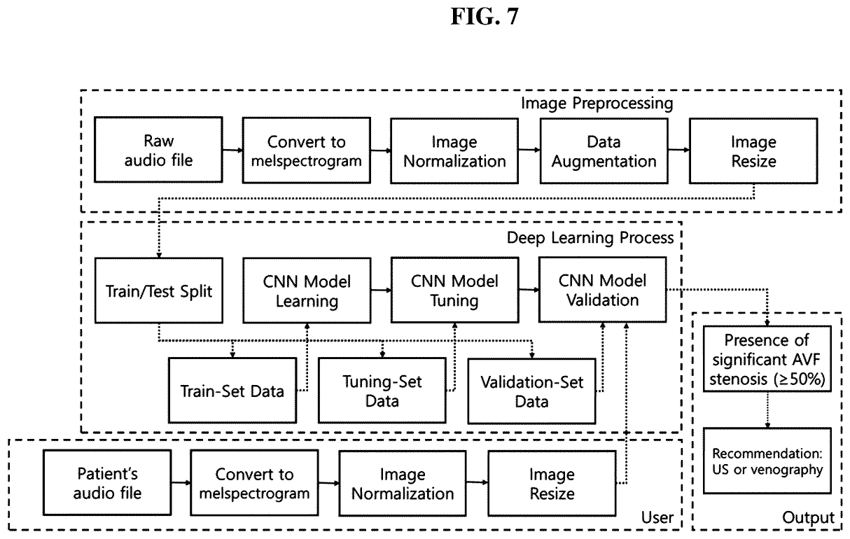

Now, an example and a performance of a method for predicting a stenosis of a dialysis access by using a convolutional neural network according to a preferable embodiment of the present invention will be described with reference to FIGS. 7 to 11.

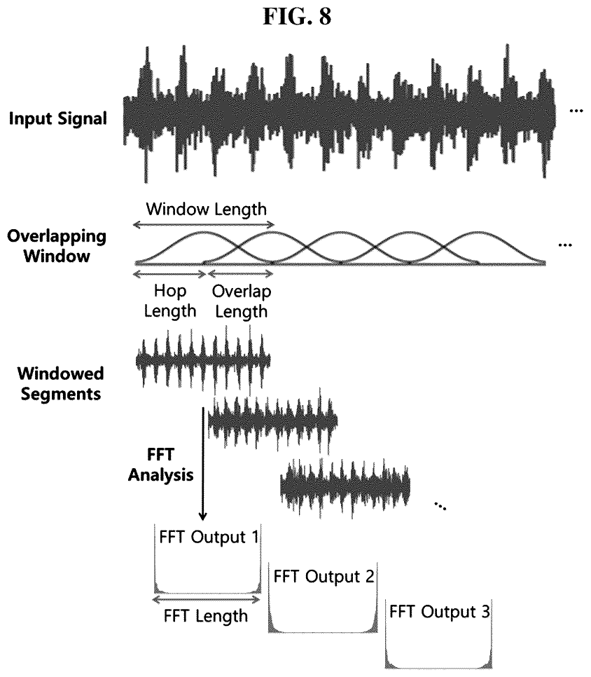

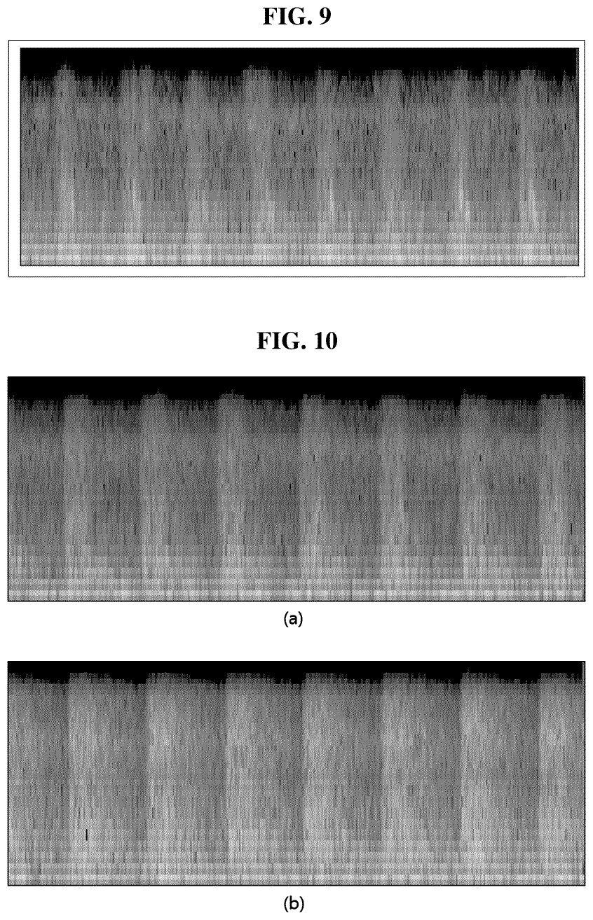

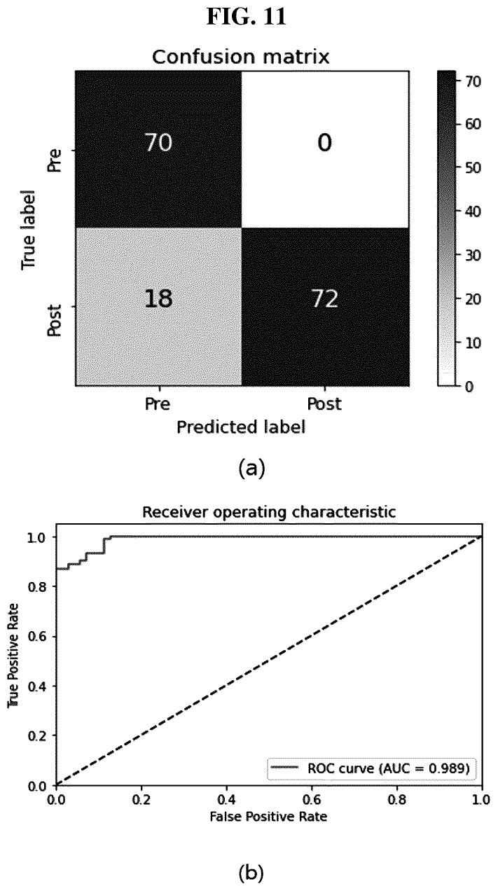

FIG. 7 is a view for explaining an example of a process of learning a stenosis prediction model and a process of predicting a degree of stenosis according to a preferable embodiment of the present invention, FIG. 8 is a view for explaining an example of a process of acquiring a spectrogram according to a preferable embodiment of the present invention, FIG. 9 is a view illustrating an example of a spectrogram acquired by the process illustrated in FIG. 8, FIG. 10 is a view illustrating an example of a spectrogram acquired by the process illustrated in FIG. 8 in which FIG. 10(a) illustrates a spectrogram acquired on the basis of audio data with respect to a dialysis access acquired before a procedure and FIG. 10(a) illustrates a spectrogram acquired on the basis of audio data with respect to a dialysis access acquired after a procedure, and FIG. 11 is a view for explaining a performance of a stenosis prediction model according to a preferable embodiment of the present invention in which FIG. 11(a) illustrates a confusion matrix and FIG. 11(b) illustrates a receiver operation characteristic (ROC) curve.

Referring to FIG. 7, an example of a method for predicting a stenosis of a dialysis access by using a convolutional neural network according to a preferable embodiment of the present invention mainly includes a stenosis prediction model learning process including an “image preprocessing process (image preprocessing illustrated in FIG. 7)” and a “deep learning process (a deep learning process illustrated in FIG. 7)” and a stenosis degree predicting process including “a deep learning process (deep learning process illustrated in FIG. 7)” and “a patient's stenosis degree predicting process (output illustrated in FIG. 7)”.

Data input to the stenosis prediction model (audio data included in a learning data set used to learn the stenosis prediction model and audio data of an object to determine a degree of stenosis) is a mel spectrogram and may be an image film having three RGB channels with a size of 512×512.

An audio file obtained by recording a sound of a dialysis access (arteriovenous fistula) of the patient is obtained using an electronic stethoscope. The recording lasted for approximately 10 seconds. However, when a person directly records, a file reproducing time of the audio file may vary depending on files and a noise, such as touching the stethoscope at the beginning and the end of the recording, may be input to the audio file. Therefore, in order to remove the noise, audio data for 6 seconds, which is the time between 2 seconds and 8 seconds, is actually used.

TABLE 1

trim_wav(DATA_DIR + fname, DATA_DIR2 + fname, 2, 8)

Thereafter, the audio file is sampled according to a specific sampling rate to store a numeric number in the form of array.

TABLE 2

y, sr = librosa.load(.wav)

Here, sr may be set as a specific value, but in the present invention, a native sampling rate is used so that sr is set to be None (sr=None). In this case, a graph of a sampling interval (x-axis, time) vs. amplitude (y-axis) may be generated, but this graph is not so useful for the analysis. The sound is basically considered as a sum of sin functions having a specific frequency and it is identified how each frequency component is constructed at a specific time by analyzing a frequency of a y waveform obtained above. This method is the Fourier transform FT. That is, when the Fourier equation is solved, a graph of amplitude vs. time is changed to a graph of frequency vs. time and in the present invention, as the Fourier transform, short time Fourier transform is used.

TABLE 3
S = librosa.feature.melspectrogram(y=y, n_mels=40, n_fft=input_nfft,
hop_length=input_stride, fmin=fmin, fmax=fmax)

Here, fmax is a maximum frequency which determines an analysis range and generally, according to Nyquist rule, the maximum frequency is determined by a value of sampling rate/2. When STFT is performed in this manner, how much divide the range to perform analysis is determined by Hop length illustrated in FIG. 8. n_fft is FFT length (or window length) to be analyzed and is determined as 25 msec. Hop length is set to 10 msec and 15 msec (overlap length) overlaps for one section.

In this manner, the mel spectrogram as illustrated in FIG. 9 may be obtained. X axis is time and Y axis is frequency and an intensity of a specific frequency in a specific time zone and decibel may be represented with colors.

However, when the learning is performed using an actual mel spectrogram, feature extraction needs to be performed. In this case, normalization of the spectrogram is necessary to improve an ability to distinguish the power of the spectrogram represented with colors in the mel spectrogram and ensure the data uniformity. That is, the levels of the noises which are inserted during the recordings may vary and a sound wave with a larger power at a specific frequency may be recorded according to the degree of stenosis. Further, in order to ensure the features are as recognizable as possible when the stenosis prediction model is trained, the spectrogram needs to be normalized.

```
TABLE 4
def normalize_mel(S):
  return np.clip((S−min_level_db)/−min_level_db, 0, 1)
def norm_mel(a):
  norm_log_S = normalize_mel(librosa.power_to_db(a, ref=np.max))
  return norm_log_S
S = librosa.feature.melspectrogram(y=y, n_mels=40, n_fft=input_nfft,
  hop_length=input_stride, fmin=fmin, fmax=fmax)
S_re = norm_mel(S)
```

In this manner, the mel spectrogram may be obtained from each audio file and examples of the spectrogram before and after the angioplasty procedure are as illustrated in FIG. 10. FIG. 10(a) is a mel spectrogram before the angioplasty procedure and FIG. 10(b) is a mel spectrogram after the angioplasty procedure. It is confirmed that after the procedure, as the degree of stenosis of the dialysis access (arteriovenous fistula) is improved, the spectrogram with a larger power at a higher frequency is seen. In fact, it is confirmed that the sound after the procedure is louder and more audible by listening to the sound recorded before and after the procedure. When the mel spectrogram was obtained before the procedure of the angioplasty, the degree of stenosis is 50% or higher (calculated by comparing diameters of a stenosis portion of the arteriovenous fistula and a healthy blood vessel during the actual venography) so that the mel spectrogram was labeled with “pre(1)” which was the first correct answer label to be stored in a folder. When the mel spectrogram was obtained after the procedure of the angioplasty, the degree of stenosis was less than 50% (when it was confirmed that the degree of stenosis is less than 50% during the actual venography) so that the mel spectrogram was labeled with “post(0)” which is the second correct answer label to be stored in a folder.

As illustrated in FIG. 9, there is a white boundary surrounding the border region of the mel spectrogram. A blue border line is arbitrarily displayed to make the boundary more obvious.

In order to train the stenosis prediction model, a number of data of mel spectrogram needs to be increased and in this case, a horizontal shifting method is used. When a convolutional neural network to recognize cats is developed, the learning is performed by assigning a plurality of angles to an original cat photo or amplifying the photo using vertical/horizontal flip techniques. In contrast, unlike the normal cat photo, the mel spectrogram is a vector gram in which x-axis, y-axis, and z-axis values and meanings are determined in advance so that the only method for increasing the data is a horizontal shift method. The data obtained by the horizontal shifting method may be used as learning data because it is a result of recording the same patient with different recording start and end times in real life. That is, the horizontal shifting is used because the mel spectrogram may be shifted along the x-axis (=time) according to the recording start and end times in the real life. When repeated peaks shown from the mel spectrogram are considered as a function of sin(x) or cos(x), a captured wave may look like sin(x+a) or cos(x+a) depending on how to set the recording time range.

The data increment uses ImageDataGenerator as follows.

```
TABLE 5
from keras.preprocessing.image import ImageDataGenerator, array_to_img,
img_to_array, load_img
data_aug_gen = ImageDataGenerator(rescale=1./255,
        rotation_range= 0,
        width_shift_range=0.9,
        height_shift_range= 0,
        shear_range=0,
        #zoom_range=[0.8, 2.0],
        horizontal_flip= False,
        vertical_flip= False,
        fill_mode=‘wrap’)
# “for” will be infinitely repeated so that a desired number of iterations is specified so
that when it reaches the specified number of iterations, exit the loop.
  for batch in data_aug_gen.flow(x, batch_size=1, save_to_dir =DATA_DIR9,
save_prefix=‘aug’, save_format=‘png’):
    I += 1
    if I > 50:
      break
```

From this code, it is understood that a width shift range is set to 0.9 for horizontal shifting. In this code, the increment is set to 50 times (i>50), that is, 50 images are generated by shifting one mel spectrogram image on the x-axis. In this case, 50 is an arbitrary value so that it is not limited to 50 times. However, as illustrated in FIG. 9, the presence of a white border area causes a white vertical line at a left or right end to be inserted during the horizontal shift to harm the mel spectrogram data so that preprocessing is performed to remove the white border area.

```
TABLE 6
#trim image to remove white border (no need to designate white 225 225
225, uses pixel (0,0))
def trim(f):
  bg = Image.new(f.mode,f.size, f.getpixel((0,0)))
  diff = ImageChops.difference(f, bg)
  diff = diff = ImageChops.add(diff, diff, 2.0, −100)
  bbox = diff.getbbox( )
  if bbox:
    return im.crop(bbox)
```

A black line which is not seen at the left and right ends of the white border area is also removed.

TABLE 7

```
#crop sides to remove streaky vertical line
def crpim(im):
  width, height = im.size
  img = im
  img_res = img.crop((10,0,width−10,height))
  return img_res
```

When the mel spectrogram obtained as described above is identified by the following method, the size is 2328×909.

TABLE 8

import cv2
im = cv2.imread(DATA_DIR6 + ‘postex.wav.png’)
h, w, c = im.shape
print(‘width: ’, w)
print(‘height: ’, h)
print(‘channel:’, c)
width: 2328
height: 909
channel: 3

If the image size is too large, there is a possibility that when the stenosis prediction model compresses the array step by step, too large area of the starting image is compressed so that the values of the mel spectrogram before and after the procedure do not show a significant difference at the last layer. Further, it takes long time to train the stenosis prediction model so that a rectangular image is adjusted to a square photo.

```
TABLE 9
img_width, img_height = 512, 512
#512 x 512 upper limit?
img_channel = 3
img_shape = (img_width, img_height, img_channel)
n_classes = 2
epochs = 10
batch_size = 15
def read_img(img_file_path, height = img_height, width = img_width):
  tmp_img = imageio.imread(img_file_path)
  tmp_img = tmp_img[:,:,:3] # get rgb channels
  tmp_img = tmp_img.astype(‘float32’) #change data type
  tmp_img −= np.min(tmp_img)
  tmp_img /= np.max(tmp_img)
  tmp_img = cv2.resize(tmp_img, (width, height), interpolation =
cv2.INTER_CUBIC)
  return tmp_img
```

This code is an example that the size of the mel spectrogram is adjusted to 512×512 and the mel spectrogram is input to the stenosis prediction model after adjusting the size.

The learning data set is divided into a training data set, a tuning data set, and a validation data set at a ratio of 7:1:2 to perform the analysis. To this end, train_test_split function is used and train_test_split randomly divides an array for a file list in the folder into training, tuning, and validation subsets.

TABLE 10

ratio_train = 0.70
ratio_tune = 0.10
ratio_val = 0.20
filelist_pre = os.listdir(PRE_PATH)
filelist_post = os.listdir(POST_PATH)
Y_pre = np.zeros(len(filelist_pre))
Y_post = np.ones(len(filelist_post))
filelist_train_tune, filelist_val, Y_train_tune, Y_val =
train_test_split(filelist, Y, stratify = Y, test_size = ratio_val,
random_state=SEED)
filelist_train, filelist_tune, Y_train, Y_tune =
train_test_split(filelist_train_tune, Y_train_tune, stratify =
Y_train_tune, test_size = (ratio_tune/(1−ratio_val)),
random_state=SEED)

Accordingly, in filelist_tune, the following melspectrogram.png files are input in the form of array.

```
TABLE 11
print(filelist_tune)
[‘aug_0_4960.png’ ‘aug_0_1335.png’ ‘aug_0_4483.png’ ‘aug_0_2761.png’
  ‘aug_0_6022.png’ ‘aug_0_460.png’ ‘aug_0_8006.png’ ‘aug_0_8392.png’
  ‘aug_0_8322.png’ ‘aug_0_1380.png’ ‘aug_0_9992.png’ ‘aug_0_8180.png’
  ‘aug_0_4821.png’ ‘aug_0_9618.png’ ‘aug_0_5742.png’ ‘aug_0_4492.png’
  ‘aug_0_6548.png’ ‘aug_0_9888.png’ ‘aug_0_2143.png’ ‘aug_0_7711.png’
  ‘aug_0_6265.png’ ‘aug_0_483.png’ ‘aug_0_6907.png’ ‘aug_0_2448.png’
  ‘aug_0_9725.png’ ‘aug_0_5616.png’ ‘aug_0_1087.png’ ‘aug_0_5973.png’
  ‘aug_0_813.png’ ‘aug_0_6349.png’ ‘aug_0_8544.png’ ‘aug_0_9848.png’
  ‘aug_0_5402.png’ ‘aug_0_159.png’ ‘aug_0_9178.png’ ‘aug_0_4356.png’
  ‘aug_0_7508.png’ ‘aug_0_6779.png’ ‘aug_0_2304.png’ ‘aug_0_4412.png’
  ‘aug_0_8247.png’ ‘aug_0_3615.png’ ‘aug_0_9967.png’ ‘aug_0_6395.png’
  ‘aug_0_7328.png’ ‘aug_0_6707.png’ ‘aug_0_8903.png’ ‘aug_0_921.png’
  ‘aug_0_1947.png’ ‘aug_0_7142.png’ ‘aug_0_5883.png’ ‘aug_0_217.png’
  ‘aug_0_3444.png’ ‘aug_0_7394.png’ ‘aug_0_1708.png’ ‘aug_0_8178.png’
  ‘aug_0_1137.png’ ‘aug_0_4933.png’ ‘aug_0_4119.png’ ‘aug_0_403.png’
  ‘aug_0_4120.png’ ‘aug_0_6206.png’ ‘aug_0_3864.png’ ‘aug_0_8954.png’
  ‘aug_0_2758.png’ ‘aug_0_4700.png’ ‘aug_0_1780.png’ ‘aug_0_8847.png’
  ‘aug_0_642.png’ ‘aug_0_9361.png’ ‘aug_0_7775.png’ ‘aug_0_4778.png’
  ‘aug_0_6093.png’ ‘aug_0_1316.png’ ‘aug_0_374.png’ ‘aug_0_7731.png’
  ‘aug_0_6636.png’ ‘aug_0_9439.png’ ‘aug_0_7850.png’ ‘aug_0_8797.png’]
```

When pre=0 and post=1, the files are a binary array as follows.

TABLE 12

```
print(Y_tune)
[0. 1. 1. 1. 0. 0. 0. 1. 1. 0. 1. 1. 0. 0. 1. 1. 0. 0. 0. 1. 1. 1. 1. 0.
  1. 1. 1. 1. 0. 0. 0. 1. 0. 1. 0. 0. 1. 0. 1. 1. 0. 1. 1. 1. 0. 0. 1. 0.
  1. 1. 1. 1. 0. 0. 1. 1. 0. 1. 1. 0. 0. 1. 0. 1. 0. 1. 1. 0. 0. 1. 1. 0.
  1. 1. 1. 1. 1. 0. 0. 0.]
```

aug_0_4960.png included in the file list in the tuning was pre (a sound obtained before the procedure) so that aug_0_4960.png was stored as 0 and aug_0_1335.png was post (a sound obtained after the procedure) so that aug_0_1335.png was stored in the array as 1. The performance of the stenosis prediction model according to the present invention was tested using ResNET50 model which is a convolutional neural network (CNN) model.

ResNET50 is configured by an input layer, a convolution layer, a max pooling layer, an average pooling layer, and an output layer, like a general convolutional neural network (CNN) model. Here, the convolution layer is formed of 50 layers to extract image features from the mel spectrogram. The max pooling layer subsamples a feature extracted from the convolution layer to increase system safety and efficiency. The average pooling layer reduces a number of parameters. The output layer outputs the following value.

That is, a value output by the output layer is a value of a prediction ability and a diagnostic performance of the stenosis prediction model for 50% or higher of stenosis of the dialysis access. For example, like the following example, values for the sensitivity, the specificity, a positive predictive value, a negative predictive value, an accuracy may be output. A confusion matrix and a receiver operation characteristic (ROC) curve as illustrated in FIG. 11 may be obtained on the basis thereof and an area under the curve (AUC) value of a diagnostic power may be calculated from the ROC curve.

TABLE 13

TN = 70 / FP = 0
FN = 18 / TP = 72
sensitivity: 80.0%
specificity: 100.0%
Accuracy >> 88.75%

YES/NO answer may be obtained as to whether 50% or more of a dialysis access (arteriovenous fistula) should be suspected from the mel spectrogram of a specific patient included in the validation data set. When the stenosis prediction model is ran, as an output, 0 or 1 is displayed for every mel spectrogram to indicate whether the stenosis is less than 50% or 50% or higher.

TABLE 14

[‘aug_0_8169.png’]
[0.94346315]

ResNet50 model performs the learning to minimize H(x)−x to make an output value of the network x so that the output is “0.94346315” which is close to 1. In this case, it is considered that the stenosis is 50% or higher. In this case, when the model is ran, the result is printed out as YES. In contrast, in the following case, the value is close to 0 so that it is recognized that the stenosis is 0 or less than 50%, rather than the stenosis of 50% or higher and the result is printed out as NO.

TABLE 15

[‘aug_0_8464.png’]
[0.05653682]

If a significant stenosis of 50% or higher is suspected, an additional examination for the stenosis of the dialysis access (arteriovenous fistula) will be recommended and in the case of YES, recommendations such as “significant stenosis of the hemodialysis access is suspected and it may result in poor hemodialysis. Additional examinations, such as Doppler ultrasound or venography are requested, so that please visit close hospital.” may be output together. When 50% or more of a dialysis access (arteriovenous fistula) is suspected from the mel spectrogram of a specific patient included in the validation data set, how much the stenosis prediction model suspects may be output as a % value.

TABLE 16

[‘aug_0_8169.png’]
[0.94346315]

The mel spectrogram of this patient means that the stenosis prediction model predicts 50% or more of stenosis with 94% of possibility. The learning process, the tuning process, and the validation process of the stenosis prediction model using ResNet50 model represented as the following codes are as follows.

TABLE 17

base_model = ResNet50(weights=None, include_top=True,
input_shape=img_shape)
output = tf.keras.layers.Dense(n_classes, activation=‘softmax’
name=‘final_layer’)(base_model.output)
model = tf.keras.models.Model(inputs=[base_model.input],
outputs=[output])
model.summary( )
n_classes = 2
epochs = 10
batch_size = 20

batch_size is a number of samples used to train a sample once and epoch is how many times to go back and forth between 50 layers of ResNet to perform learning. That is, epochs are 10, it means that the learning is performed using premise data 10 times. If these values are not fixed, batch_size and the epoch value need to be modified several times to optimize the model. When the epoch value is too small, the model has a tendency of underfitting to the data and when the epoch value is too large, the model has an overfitting problem. For example, when there are 100 mel spectrograms, a batch size is 20 so that 20 data is learned at every iteration so that 1 epoch=100/batch size=5 iterations. Therefore, if there are 40 epochs, 200 iterations are performed.

An optimizer used to update the model at every learning is Keras SGD (stochastic gradient descent) as follows. There are also many types of optimizes, such as RMSprop, Adam, and Adadelta. Even though in the present invention, SGD was used, but the optimizer is not limited to SGD. When the stenosis prediction model is trained, generally, as a learning rate, a value of 0.01 to 0.1 is used and momentum is set to 0.9 in many cases. In the present invention, the learning rate is set to 0.02.

TABLE 18
##optimizer and loss##
opt = SGD(learning_rate=0.02, momentum=0.9, decay=1e−2/epochs)
metrics = [‘accuracy’]
model.compile(loss=‘binary_crossentropy’, optimizer=opt,
metrics=metrics)

The basic principle of optimizing is to make the learning rate “bigger at first, then smaller” (see Reference Document: Qian Ning, On the momentum term in gradient descent learning algorithms, Neural networks 12.1 (1999) 145-151). The momentum uses the same value of the learning rate and when the parameter is changed, an adjustment term called the momentum term is used to similarly express the concept of “bigger at first, then smaller”.

When a parameter of a neural network model for an error function E is θ, a gradient of E with respect to θ is ∇θE, and a difference of the parameter Δθ(t) is Equation (1), an equation of changing the parameter using the momentum at the step t is Equation (2).
γΔθ^{(t-1)}: Momentum Term
Coefficient γ(<1) generally sets a value of 0.5 or 0.9.

$$Δ {θ}^{( t )} = Δ {θ}^{( t )} - {γΔθ}^{( t - 1 )} \qquad ( 1 )$$

$$Δ {θ}^{( t )} = - η {∇}_{θ} E ( θ ) + {γΔθ}^{( t - 1 )} \qquad ( 2 )$$

That is, when a learning rate and a momentum are set as described above, at an initial epoch, the accuracy is increased quickly.

All the parameters are set as described above and the stenosis prediction model is fitted or trained as follows.

```
TABLE 19
n_points = len(filelist_train) #number of train data (string length)
nb_tune_samples = len(filelist_tune) # number of tune data
model_history = model.fit(generator_train_fx( ),
            steps_per_epoch = n_points // batch_size,
            epochs=epochs,
            verbose=1,
            callbacks=callbacks_list,
            validation_data=generator_tune_fx( ),
            validation_steps = nb_tune_samples // batch_size)
def generator_train_fx( ):
  while True:
    for i in range(len(filelist_train) // batch_size): #step
      batch_img = np.zeros((batch_size, img_height, img_width,
img_channel))
      batch_smk = np.zeros((batch_size, 2), dtype=np.float16)
      for j in range(batch_size): #batch size
        filename = filelist_train[i*batch_size+j]
        label = Y_train[i*batch_size+j]
        img = read_img(get_img_path(filename, label), img_height,
img_width)
        if label == 1.0: #post
          batch_smk_tmp=[1., 0.]
        elif label == 0.0: #pre
          batch_smk_tmp=[0., 1.]
        batch_img[j] = img
        batch_smk[j] = batch_smk_tmp
      yield batch_img, batch_smk
```

The mel spectrograms are separately stored by pre and post files so that when it is called from PRE_PATH+filename, it is trained with 1 and when it is called from POST_PATH+filename, it is trained with 0 to create the model. During the tuning or fine tuning step, a model having a highest accuracy for tuning-set data is chosen based on an accuracy of a model completed at every epoch seen when the tuning-set data is input. For example, as for text, epochs are set to 10 and the model is ran, the following results may be obtain.

TABLE 20
Epoch 1/10
27/27 [==============================] - 49s 1s/step - loss: 0.6919 - accuracy:
0.5981 - val_loss: 0.6877 - val_accuracy: 0.5625
Epoch 2/10
27/27 [==============================] - 36s 1s/step - loss: 0.6827 - accuracy:
0.5981 - val_loss: 0.6857 - val_accuracy: 0.5625
Epoch 3/10
27/27 [==============================] - 36s 1s/step - loss: 0.6713 - accuracy:
0.5981 - val_loss: 0.6853 - val_accuracy: 0.5625
Epoch 4/10
27/27 [==============================] - 37s 1s/step - loss: 0.6296 - accuracy:
0.7584 - val_loss: 0.6860 - val_accuracy: 0.5625
Epoch 5/10
27/27 [==============================] - 37s 1s/step - loss: 0.5501 - accuracy:
0.9664 - val_loss: 0.6908 - val_accuracy: 0.5625
Epoch 6/10
27/27 [==============================] - 37s 1s/step - loss: 0.4679 - accuracy:
0.9205 - val_loss: 0.7316 - val_accuracy: 0.5625
Epoch 7/10
27/27 [==============================] - 37s 1s/step - loss: 0.3894 - accuracy:
0.9239 - val_loss: 0.7642 - val_accuracy: 0.5625
Epoch 00007: ReduceLROnPlateau reducing learning rate to 0.009999999776482582.
Epoch 8/10
27/27 [==============================] - 37s 1s/step - loss: 0.3528 - accuracy:
0.9278 - val_loss: 0.4207 - val_accuracy: 0.8500
Epoch 9/10
27/27 [==============================] - 37s 1s/step - loss: 0.2593 - accuracy:
0.9831 - val_loss: 0.3553 - val_accuracy: 0.9000
Epoch 10/10
27/27 [==============================] - 37s 1s/step - loss: 0.2822 - accuracy:
0.9471 - val_loss: 0.5197 - val_accuracy: 0.8000

Here, accuracy is an accuracy of a model for training set and val_accuracy is an accuracy of a model for the training-set. As described above, a small epoch value causes an underfitting problem and a large epoch value causes an overfitting problem. Accordingly, from epoch 1/10 to epoch 10/10, it is understood that the accuracy is improved (0.5981->0.9471), but val_accuracy for tuning-set has a peak at epoch 9/10 and is slightly reduced to 0.0800 at epoch 10/10. The reason is that due to overfitting to the training set melspectrogram, when the tuning set mel spectrogram is input, the accuracy is lowered due to the poor fitting. Accordingly, in the tuning step, a model of an epoch with the best accuracy and val_accuracy is determined. In the above-example, Epoch 9/10 model is determined to be tuned. Accordingly, after determining Epoch 9 train weight, the model is applied to the validation set to find out how well it makes prediction.

TABLE 21
model.load_weights(‘/content/drive/My
Drive/AVFstudy/weights/20210112412/train_weights_epoch_009.h5’)
#change the file directory of the selected weights
Y_pred = model.predict(generator_validation_fx( ),
steps=len(filelist_val)//batch_size + 1)
Y_pred = Y_pred[: len(filelist_val),:]
print(filelist_val)
print(Y_pred)

Accordingly, the above-described output value may be obtained.

The operation according to the preferable embodiment of the present disclosure may be implemented as a program instruction which may be executed by various computers to be recorded in a computer readable storage medium. The computer readable storage medium indicates an arbitrary medium which participates to provide a command to a processor for execution. The computer readable storage medium may include solely a program command, a data file, and a data structure or a combination thereof. For example, the computer readable medium may include a magnetic medium, an optical recording medium, and a memory. The computer program may be distributed on a networked computer system so that the computer readable code may be stored and executed in a distributed manner. Functional programs, codes, and code segments for implementing the present embodiment may be easily inferred by programmers in the art to which this embodiment belongs.

The present embodiments are provided to explain the technical spirit of the present embodiment and the scope of the technical spirit of the present embodiment is not limited by these embodiments. The protection scope of the present embodiments should be interpreted based on the following appended claims and it should be appreciated that all technical spirits included within a range equivalent thereto are included in the protection scope of the present embodiments.

### EXPLANATION OF REFERENCE NUMERALS AND SYMBOLS

100: Stenosis predicting device
110: Processor
130: Computer readable storage media
131: Program
150: Communication bus
170: Input/output interface
190: Communication interface

1. A method for predicting a stenosis of a dialysis access by using a convolutional neural network, comprising:
acquiring audio data with respect to a dialysis access of an object; and
predicting a degree of stenosis corresponding to the audio data on the basis of a stenosis prediction model including a previously learned convolutional neural network (CNN).

2. The method for predicting a stenosis of a dialysis access of claim 1, wherein the acquiring of audio data is configured by preprocessing the audio data and
the predicting of a degree of stenosis is configured by inputting the preprocessed audio data to the stenosis prediction model and predicting a degree of stenosis corresponding to the audio data on the basis of an output value of the stenosis prediction model.

3. The method for predicting a stenosis of a dialysis access of claim 2, wherein the acquiring of audio data is configured by acquiring audio data in a predetermined interval of the audio data, acquiring a spectrogram on the basis of the audio data in the predetermined interval, normalizing the acquired spectrogram, and adjusting a size of the normalized spectrogram.

4. The method for predicting a stenosis of a dialysis access of claim 1, further comprising:
learning the stenosis prediction model on the basis of a learning data set including first audio data with respect to the dialysis access acquired before a procedure and second audio data with respect to the dialysis access acquired after the procedure.

5. The method for predicting a stenosis of a dialysis access of claim 4, wherein the stenosis prediction model has the spectrogram as an input and a degree of the stenosis as an output.

6. The method for predicting a stenosis of a dialysis access of claim 5, wherein the learning of the stenosis prediction model is configured by preprocessing the learning data set and learning the stenosis prediction model on the basis of a learning data set preprocessed with first audio data as a first correct answer label and second audio data as a second correct answer label.

7. The method for predicting a stenosis of a dialysis access of claim 6, wherein the learning of the stenosis prediction model is configured by acquiring the audio data in a predetermined interval from the audio data, with respect to audio data included in the learning data set, acquiring a spectrogram on the basis of the audio data in the predetermined interval, normalizing the acquired spectrogram, horizontally shifting the normalized spectrogram to increase the number, and adjusting the size of the increased spectrogram to preprocess the learning data set.

8. The method for predicting a stenosis of a dialysis access of claim 6, wherein the learning of the stenosis prediction model is configured by dividing the preprocessed learning data set into a training data set, a tuning data set, and a validation data set according to a predetermined criteria, learning the stenosis prediction model by using the training data set, tuning the learned stenosis prediction model using the tuning data set, and validating the tuned stenosis prediction model using the validation data set.

9. A computer program stored in a computer readable storage medium to allow a computer to execute the method for predicting a stenosis of a dialysis access by using a convolutional neural network of claim 1.

10. A device for predicting a stenosis of a dialysis access by using a convolutional neural network (CNN), comprising:
a memory which stores one or more programs to predict a stenosis of a dialysis access using a convolutional neural network (CNN); and
one or more processors which perform an operation for predicting a stenosis of a dialysis access using a convolutional neural network (CNN) according to one or more program stored in the memory,
wherein the processor acquires audio data with respect to a dialysis access of an object and predicts a degree of stenosis corresponding to the audio data on the basis of the stenosis prediction model including a previously learned convolutional neural network (CNN).

11. The device for predicting a stenosis of a dialysis access of claim 10, wherein the processor preprocesses the audio data, inputs the preprocessed audio data to the stenosis prediction model, and predicts a degree of stenosis corresponding to the audio data on the basis of an output value of the stenosis prediction model.

12. The device for predicting a stenosis of a dialysis access of claim 10, wherein the processor learns the stenosis prediction model on the basis of a learning data set including first audio data with respect to the dialysis access acquired before a procedure and second audio data with respect to the dialysis access acquired after the procedure.

13. The device for predicting a stenosis of a dialysis access of claim 12, wherein the processor preprocesses the learning data set and learns the stenosis prediction model on the basis of the learning data set preprocessed with first audio data as a first correct answer label and second audio data as a second correct answer label.
